(19) United States
(12) Patent Application Publication
(10) Pub. No.: US 2023/0397846 A1
(43) Pub. Date: Dec. 14, 2023

(54) WEARABLE DEVICE FOR SENSING CHLORIDE IN SWEAT

(71) Applicant: TWENTY TWENTY THERAPEUTICS LLC, South San Francisco, CA (US)

(72) Inventors: Zidong Li, South San Francisco, CA (US); Fred Lee, South San Francisco, CA (US); Dimitri Azar, South San Francisco, CA (US)

(21) Appl. No.: 18/208,124

(22) Filed: Jun. 9, 2023

Related U.S. Application Data
(60) Provisional application No. 63/350,444, filed on Jun. 9, 2022.

(51) Int. Cl. A61B 5/145; A61B 10/00; A61B 5/1477; A61B 5/00; G01N 27/30

(52) U.S. Cl. CPC A61B 5/14521; A61B 10/0064; A61B 5/14546; A61B 5/1477; A61B 5/4839; A61B 5/0004; G01N 27/30; A61N 1/0432

(57) ABSTRACT

A wearable device can include a sweat collection unit, and optionally, a sweat stimulation unit. The sweat collection unit includes at least one microfluidics channel to collect sweat from a user, a chamber to hold the sweat, and a loading chamber with sweat collection paper that attracts the sweat through the chamber. A chloride sensing unit located within the chamber includes a gold working electrode and a counter electrode that can determine if enough sweat is in the chamber and, if enough, the chloride concentration of the sweat can be determined based on electrochemical reactions in response to electrical signals applied through the electrodes. A controller of the device can notify a user to trigger the sweat stimulation unit if not enough sweat is in the chamber.

### CROSS-REFERENCE TO RELATED APPLICATIONS

This application claims the benefit of U.S. Provisional Application Ser. No. 63/350,444, filed 9 Jun. 2022, entitled “WEARABLE DEVICE FOR SENSING CHLORIDE IN SWEAT”. The entirety of this provisional application is incorporated by reference for all purposes.

### TECHNICAL FIELD

The present disclosure relates to evaluating and/or monitoring a physiological condition of a user, and, more specifically, to a wearable device for sensing a chloride concentration in sweat to evaluate and/or monitor the physiological condition of the user.

### BACKGROUND

Chloride concentrations in sweat can be biomarkers for many diseases and disorders, such as cystic fibrosis, electrolyte imbalance, dehydration, and the like. The gold standard for screening and diagnosis of cystic fibrosis is sweat testing, but the traditional sweat test is complex, utilizes bulky equipment, and takes several days to receive results. For cystic fibrosis, early diagnosis and treatment is paramount. Delays in receiving results may be harmful to those needing early intervention and the bulk and complexity of the entire sweat test procedures make diagnosis extremely inconvenient. Additionally, due to the complexity of the sweat test, the sweat sample volume and/or quality may not be good enough to generate accurate chloride measurements. Moreover, when testing for electrolyte imbalances and/or dehydration quick and accurate chloride concentration results are important for accurately diagnosing and properly treating a person before their condition worsens. Recently, single use devices have been developed to detect chloride concentration, but such single use devices are complicated to manufacture and are expensive to use.

### SUMMARY

There is a need for a reusable wearable device that is easy to manufacture and use and can sense a chloride concentration in sweat quickly and accurately. Provided herein are systems and methods that employ a sweat collection unit of such a wearable device to sense the chloride concentration in sweat to evaluate and/or monitor health of the user. Optionally, if the sweat collection unit cannot collect enough sweat to detect a chloride concentration independently, then a sweat stimulation unit of the wearable device can be activated to induce the user to sweat.

In one aspect, the present disclosure includes a flexible, wearable device comprising at least one microfluidics channel with hydrophilic surfaces that can collect sweat from a user's skin, a chamber to hold the sweat, and a loading chamber comprising disposable sweat collection paper that attracts the sweat so the chamber is continuously filling with fresh sweat. A chloride sensing unit located within the chamber comprises a gold working electrode and a counter electrode that can determine whether the sweat is within the chamber and when the sweat is within the chamber, deliver a voltage waveform that causes chloride ions in the sweat to undergo an electrochemical reaction with gold molecules of the gold working electrode. Signals reflecting the electrochemical reactions between the chloride ions and the gold molecules can be transmitted to an external device that can notify a user of a condition based on the signals.

In another aspect, the present disclosure includes a system comprising a controller that can set parameters for a plurality of voltage waveforms, a signal generator coupled to the controller that can generate the plurality of voltage waveforms, and a sweat sensing and collection unit. The sweat sensing and collection unit comprises at least one inlet having microfluidic properties that can collect sweat when positioned on skin of user using negative capillary pressure to attract the sweat, a sweat chamber that can hold a volume of the sweat from the inlet, a loading chamber in fluid communication with the sweat chamber, and at least one microchannel in fluid communication with the at least one inlet, the sweat chamber, and the loading chamber. The sweat chamber comprises a gold working electrode and a counter electrode that can deliver one of the plurality of voltage waveforms therebetween so that the controller can determine whether a sufficient volume of sweat is within the sweat chamber based on an impedance measured between the gold working electrode and the counter electrode. When the sufficient volume of sweat is within the chamber, the gold working electrode and the counter electrode can deliver another voltage waveform of the plurality of voltage waveforms that can cause chloride ions in the sweat to undergo an electrochemical reaction with gold molecules in the gold working electrode so that the controller can determine the chloride concentration of the sweat within the chamber based on the electrochemical reaction. The loading chamber that is in fluid communication with the sweat chamber can hold at least a portion of the volume of sweat from the sweat chamber and can include a sweat attracting mechanism that can continuously draw sweat from the sweat chamber into the loading chamber. The at least one microchannel can use negative capillary pressure to move sweat from the at least one inlet to the sweat chamber and to the loading chamber.

In another aspect, the present disclosure includes a method for sensing an amount of sweat in a chamber of a sweat collection device and determining a concentration of chloride in the sweat in the chamber if enough sweat is present. The method includes determining, by a system comprising a processor, whether an amount of sweat fills a chamber of a sweat collection unit, where a chloride sensing unit that comprises a gold working electrode and a counter electrode is located within the chamber and the determining is based on an impedance between the gold working electrode and the counter electrode. When the impedance indicates that a sufficient amount of sweat fills the chamber the method also includes delivering, by the system, a voltage waveform between the gold working electrode and the counter electrode within the chamber where molecules from the gold working electrode electrochemically react to the sweat in the chamber. The method also includes detecting, by the system, a chloride concentration of the sweat in the chamber based on signals associated with the electrochemical reaction between the chloride ions and the molecules from the gold working electrode. Information regarding the chloride concentration can be used to determine more physiological conditions of the user based on the chloride concentration of the sweat in the chamber.

### BRIEF DESCRIPTION OF THE DRAWINGS

The foregoing and other features of the present disclosure will become apparent to those skilled in the art to which the present disclosure relates upon reading the following description with reference to the accompanying drawings, in which:

FIG. 1 shows a block diagram of a sweat collection unit and other components of a wearable device for collecting and detecting chloride in sweat;

FIG. 2 shows a block diagram of the sweat collection unit of the of the wearable device of FIG. 1;

FIG. 3 shows a block diagram of the components of the wearable device and an external device in communication with the wearable device;

FIG. 4 shows a diagrammatic representation of the sweat stimulation unit of the wearable device of FIG. 3;

FIG. 5 shows an example implementation of the sweat collection and sweat stimulation units of the wearable device of FIG. 3;

FIG. 6 shows example implementations of the wearable device as a patch or a bracelet/watch;

FIG. 7 shows illustrations of the top, middle, and bottom layers of an example implementation of the wearable device of FIG. 3 having a flexible substrate;

FIGS. 8-15 show an example use of the exemplary implementations of the sweat stimulation unit and the sweat collection unit when the wearable device is positioned on a user's skin;

FIGS. 16 and 17 show example process flow diagrams of methods for determining a chloride concentration of sweat of a user;

FIG. 18 shows an example process flow diagram of a method for notifying a user of the wearable device of a physiological condition based on chloride concentration;

FIG. 19 shows an example process flow diagram of a method that can be employed by the control logic for determining if the sweat stimulation unit should be activated, activating the sweat stimulation unit, and activating the sweat collection unit.

### DETAILED DESCRIPTION

### I. Definitions

Unless otherwise defined, all technical terms used herein have the same meaning as commonly understood by one of ordinary skill in the art to which the present disclosure pertains.

As used herein, the singular forms “a,” “an,” and “the” can also include the plural forms, unless the context clearly indicates otherwise.

As used herein, the terms “comprises” and/or “comprising,” can specify the presence of stated features, steps, operations, elements, and/or components, but do not preclude the presence or addition of one or more other features, steps, operations, elements, components, and/or groups.

As used herein, the term “and/or” can include any and all combinations of one or more of the associated listed items.

As used herein, the terms “first,” “second,” etc. should not limit the elements being described by these terms. These terms are only used to distinguish one element from another. Thus, a “first” element discussed below could also be termed a “second” element without departing from the teachings of the present disclosure. The sequence of operations (or acts/steps) is not limited to the order presented in the claims or figures unless specifically indicated otherwise.

As used herein, the terms “sweat” and “perspire” are verbs that can refer to the production of fluids secreted by the sweat glands in the skin of a subject.

As used herein, the terms “sweat” and “perspiration” are nouns that can refer to the fluids thus secreted from the sweat glands. Sweat is mostly composed of water, but can include amounts of salts (e.g., that include chloride), proteins, urea, and/or ammonia.

As used herein, the terms “sweat collection paper” and “sweat collection mechanism” can refer to a material capable of attracting and absorbing a quantity of sweat. Such material can be porous to enhance the liquid attraction capability. The sweat collection paper may be replaceable when the sweat collection paper has absorbed a quantity of sweat.

As used herein, the term “electrode” refers to a conductive solid (e.g., including one or more metals, one or more polymers, or the like) that receives/transmits an electrical signal. For example, the device described herein can have a plurality of electrodes with different functionalities. In some instances, the device can have one or more working electrodes and one or more counter electrodes.

As used herein, the term “working electrode” refers to an electrode (e.g., a metal electrode) on which a reaction of interest (e.g., electrodissolution) is occurring. A non-limiting example of the working electrode is a thin-film gold electrode.

As used herein, the term “counter electrode” refers to an electrode that completes the circuit and applies input potential. A counter electrode can also function as both a counter electrode and a reference electrode so it may also have a constant electrochemical potential as long as no current flow through it.

As used herein, the term “electrical signal” refers to a waveform generated by an electronic means, such as a generator. An electrical signal may be a voltage signal or a current signal.

As used herein, the term “voltage” refers to a potential difference in charge between two points.

As used herein, the term “current” refers to a flow of electrical charge carriers.

As used herein, the term “electrodissolution” refers to a process for dissolving a solute using an electrical catalyst. In one non-limiting example, application of an electrical signal to a solid metal can cause the solid metal to dissolve into separate molecules.

As used herein, the term “electrochemical reaction” refers to any processes caused or accompanied by the passage of an electric current and involving in most cases the transfer of electrons between two substances. For example, when a special voltage waveform is applied between a gold working electrode and a counter electrode and chloride ions are present, then an electrochemical reaction occurs between the gold molecules of the gold working electrode and the chloride molecules. Such electrochemical reactions can be measured based on electrochemical signals (e.g., current or charge).

As used herein, the term “wearable device” refers to a device that can be removably attached to skin of a user. For example, the wearable device can be part of a wristband, part of a watch, or part of a patch attached to the skin by an adhesive. The wearable device can include, but is not limited to, components used for the detection of sweat and chloride concentrations therein and components to collect sweat. The wearable device may also include components to trigger the release of sweat.

As used herein, the term “reservoir” refers to a storehouse for a substance with a portion being open for release of the substance from the reservoir. The opening may be covered to prevent release of the substance until a certain time or action has occurred. In some instances, the covering can facilitate release of the therapeutic from the reservoir. For example, at least a portion of the covering can be an electrode that can electrodissolve to facilitate the release of the therapeutic.

As used herein, the term “drug” refers to one or more substance (e.g., liquid, solid, or gas) which has a physiological effect when introduced into the body. For example, a drug, such as pilocarpine, can induce sweating in a user when it is introduced into the body via iontophoresis.

As used herein, the terms “user” and “subject” refer to one or more wearers of the wearable device and/or a person aiding with the use of the wearable device.

As used herein, the term “iontophoresis” refers to the process of moving molecules through a permeable substance (e.g., skin, saline, etc.) by use of a voltage gradient applied to the substance by at least two electrodes. For example, iontophoresis can be used for transdermal drug delivery, where molecules can be transported across the stratum corneum by electrophoresis and electroosmosis.

As used herein, the term “microfluidics” refers to the behavior, precise control, and manipulation of fluids that are geometrically constrained to a small scale at which surface forces dominate volumetric forces. Microfluidics can be exploited in a “microfluidics channel” (also referred to as a “microchannel”) to bring fluid into at least a portion of the wearable device.

### II. Overview

Chloride concentrations in sweat can be biomarkers for many diseases and disorders, but traditional sweat tests to detect chloride concentrations in sweat tend to be complex, expensive, and often take an extended time period to receive results. Accordingly, there is a need for a sweat chloride test that allows subjects to obtain quick and accurate results (either through continuous monitoring, monitoring at given intervals, or monitoring a single time). The sweat chloride test can be embedded in a portable and wearable device, which may also include a sweat induction mechanism. Traditional sweat tests include sweat induction using gelled pilocarpine iontophoresis from a pair of bulky electrodes attached to the skin with an external power generator, sweat sample collection from a separate device placed on the skin, transportation of the sweat samples to an analytical laboratory, and then sweat chloride measurement utilizing the laboratory apparatus. Sweat induction and collection traditionally take a few hours (e.g., 1 or 2 hours, or the like), but obtaining the chloride measurement results from the laboratory can take a few days. This delay in the time to receive results may be harmful to those needing early intervention and the bulk and complexity of the entire sweat test procedure makes diagnosis extremely inconvenient. Additionally, due to the complexity of a traditional sweat test, the sweat sample volume and/or quality may not be good enough to generate accurate chloride concentration measurements. Then the sweat test would need to be re-performed and the process begun again. Moreover, sweat tests can only be performed at certified clinical centers with bulky apparatuses, making it impossible for a subject to accurately self-monitor and receive continuous monitoring during their daily lives. Another limitation to the traditional sweat test is that the pilocarpine used to induce sweat for the sweat test is preserved in a gel patch that has a limited shelf life and has to be stored in 2-10° C. Recently, there has been a push to develop novel chloride biochemical sensors and to make sweat tests easier by combining sweat collection and chloride measurement into one device. However, these chloride sensors have several limitations. First, these novel chloride biochemical sensors are complicated to manufacture and have a high cost because they require extensive preparation work on biochemical assays and/or electrode chemical modifications. Second, these chloride sensors are single-use disposable sensors and can only provide a one-time chloride measurement. Third, sweat stimulation via pilocarpine gels cannot be triggered on-demand more than once, which makes it impossible for a wearable device to provide more than one chloride measurement during the wearing time.

Described herein are systems and methods for using a wearable device to evaluate and/or monitor a physiological condition by sensing a chloride concentration in sweat. The device can include a sweat collection unit that can collect sweat and a chloride sensing unit that can sense the chloride concentration in the collected sweat based on an electrochemical reaction between an electrode of the chloride sensing unit and the chloride in the sweat when a specific voltage is applied to the chloride sensing unit. The wearable device can also include a sweat stimulation unit that can be actuated by a user and then can release a drug, such as a solid (e.g., powdered) pilocarpine, to induce the wearer of the wearable device to sweat when not enough sweat is available in the collection unit for an accurate test. The wearable device can determine if enough sweat is available and communicate with an external device (e.g., mobile phone) to notify the user when not enough sweat is available. The wearable device can be reusable and can quickly and accurately perform a sweat test on a user. Such a portable and wearable device can include at least embedded mechanisms, circuitry and control logic for pilocarpine storage and on-demand release, sweat induction, sweat collection, chloride measurement, and data uploading all from one single device.

### III. System

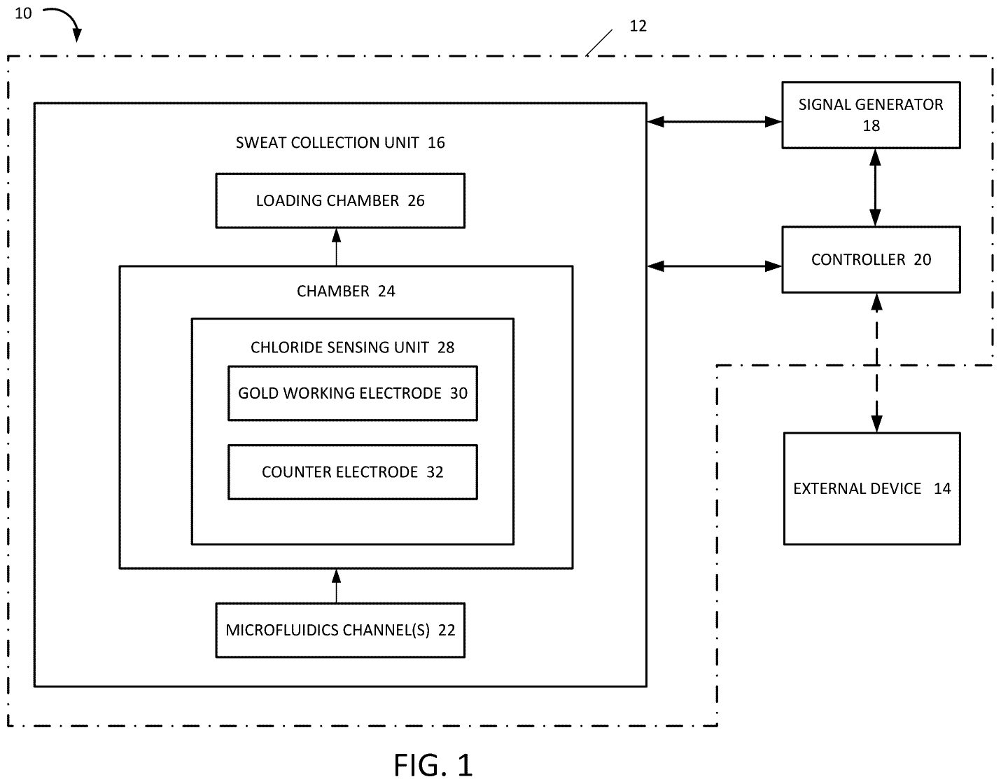

Provided herein is a system 10 (FIG. 1) that can evaluate and/or monitor a physiological condition of a user by detecting chloride and/or a chloride concentration in the sweat of a user who is wearing the wearable device 12. The system 10 can include a wearable device 12, such as a patch or an arm band, in communication with an external device 14 (e.g., a smart phone, a smart watch, a tablet, a computer, etc.). The wearable device 12 can include at least a signal generator 18, a controller 20, and a sweat collection unit 16 that can collect sweat and detect chloride and/or chloride concentrations in that sweat. Although not shown, it should be understood that the wearable device 12 can also include a wireless transceiver, a power source, and/or associated/additional circuitry. The wearable device 12 can also include a sweat stimulation unit (not shown in FIG. 1) that can induce a user to sweat if the user is not generating enough sweat for portions of the wearable device 12 to accurately detect chloride and/or a chloride concentration.

The wearable device 12 can be in communication, wireless and/or wired, with the external device 14. The external device 14 includes at least a processor and a notification mechanism to alert the user of the recorded sweat concentration (e.g., audio, tactile, and/or visual). The wearable device 12 can be flexible and can include at least a sweat collection unit 16, a signal generator 18, and a controller 20. The sweat collection unit 16 can include at least one microfluidics channel 22 that has hydrophilic surfaces configured to collect sweat from the user's skin. The microfluidics channel(s) are in fluid communication with at least a chamber 24 that can hold a given volume of the sweat collected from the user's skin. A loading chamber 26 can be in fluid communication with the chamber 24, directly or via a microfluidics channel 22, to attract sweat from the chamber so that the chamber is continuously filling with fresh sweat. The loading chamber 26 can include a disposable sweat collection paper, or another disposable material that is porous and can attract sweat, that helps to attract the sweat out of the chamber 24. The loading chamber 26 can be positioned above the chamber 24 with respect to the skin so that the older sweat (not the newly collected sweat) is attracted into the loading chamber 26.

A chloride sensing unit 28 located within the chamber can include a gold working electrode 30 and a counter electrode 32. For example, the gold working electrode 30 can be a circular working electrode and the counter electrode 32 can be a semi-circular counter electrode. The counter electrode 32 can include gold, silver chloride, and/or platinum. The gold working electrode 30 and the counter electrode 32 can be in electrical communication with the signal generator 18 and the controller 20. The signal generator 18 can generate a plurality of voltage waveforms and send at least one of the voltage waveforms to at least the gold working electrode 30. However, it should be noted that the signal generator 18 can generate and send a voltage waveform to any of the electrodes described herein. The signal generator 18 can transmit the voltage waveforms over a wired connection, a wireless connection, or a combination of wired and wireless connection. The controller 20 can be electrically coupled to the signal generator 18 and can set parameters (e.g., magnitude, timing, shape, pulsing, etc.) for the plurality of voltage waveforms generated by signal generator 18. The electrical coupling can be via a wired connection, a wireless connection, or a connection that is some combination of wired and wireless connection.

The controller 20 can store and execute instructions (e.g., computer executable instructions) related to the plurality of voltage waveforms applied to the electrodes of the wearable device 12. For example, the controller 20 can store and execute instructions for determining via the gold working electrode 30 and the counter electrode 32 if a sufficient amount of sweat is in the chamber 24 and instructions for sensing chloride and/or a chloride concentration in the sweat via the gold working electrode and the counter electrode. The controller 20 can also store and/or execute additional instructions, data, and information. For example, the controller 20 can be implemented as a type of processor. The processor can be, for example, embedded within one or more application specific integrated circuits (ASICs), digital signal processors (DSPs), digital signal processing devices (DSPDs), programmable logic devices (PLDs), field programmable gate arrays (FPGAs), microprocessors, other electronic units designed to perform the functions of a processor, or the like. The controller 20 can have a memory coupled to the processor (e.g., the functionality may be implemented by separate chips). However, in some instances the memory and the processor can be implemented together (e.g., embodied within the same chip) (e.g., a microcontroller device). Optionally, the controller 20 can be in communication (wired or wireless) with an external device comprising at least one of a display (e.g., a video screen), a memory and a processor, and an input device (e.g., a keyboard, touch screen, and/or a mouse).

The gold working electrode 30 and the counter electrode 32 can be arranged to gather information that the controller 20 can use to (1) determine whether sweat is within the chamber 24 and, when the sweat is in the chamber, (2) determine the chloride concentration in the sweat. It should be noted that the gold working electrode 30 and the counter electrode 32 can have different functionalities with the two steps.

The determination (1) of whether the sweat is within the chamber 24 is made by applying an electrical signal therebetween and measuring a resulting impedance. The impedance measured between the gold working electrode 30 and the counter electrode 32 can be used by the controller 20 to determine whether a sufficient amount of sweat is in the chamber 24. If sufficient sweat is available within the chamber 24, (e.g., covering both the gold working electrode 30 and the counter electrode 32) then the impedance measurement reflects this. If the controller 20 determines not enough sweat is available in the chamber 24 based on the impedance measurement, then the user can be notified to actuate a sweat stimulation unit (not shown), or the controller can automatically activate the sweat stimulation unit. If the controller 20 determines enough sweat is available in the chamber 24 based on the impedance measurement, then the controller can execute instructions for step (2) to determine the chloride concentration in the sweat and switch the functionality of the gold working electrode 30 and the counter electrode 32.

When sweat (e.g., a sufficient amount of sweat) is within the chamber 24 (determined based on the impedance), then the gold working electrode 30 can deliver a voltage waveform (generated by the signal generator 18) that can cause chloride ions in the sweat to undergo an electrochemical reaction with gold molecules of the gold working electrode that causes the gold to electrodissolve. The counter electrode 32 can return the electrical signal to the signal generator, completing the circuit. The voltage waveform can be one of amperometry, a linear increase square wave voltammetry, a linear step increase voltammetry, a linear ramp increase voltammetry, and a cyclic voltammetry. The electrochemical reaction of the chloride ions and the gold molecules change the signals (e.g., current or charge) that can be detected by controller 20 and/or external device 14 via at least one of the gold working electrode 30 and the counter electrode 32. Specific signals relate to specific chloride concentrations in the sweat. Detection of the chloride concentration can be done a plurality of times (e.g., 2, 10, 20, 30 times or the like) before the electrochemical reaction between the chloride ions and the gold molecules causes the gold working electrode 30 to electrodissolve beyond usefulness. The number of detections possible corresponds to the size of the gold working electrode 30 (e.g., larger electrode, more detections possible). The maximum number of detections can be decided by calculating the gold thickness of the gold working electrode 30 and the charge transfer from the gold working electrode. The calculations can be done by the controller 20 and/or the external device 14. When a maximum number of tests has been reached, the user can be notified (e.g., by sound or display from the external device 14). The impedance measurement to detect if sufficient sweat is within the chamber 24 does not cause significant damage to the gold working electrode 30. Signals reflecting the electrochemical reaction between the chloride ions and the gold molecules can be transmitted to the external device 14 (e.g., by a wireless transceiver not shown) and/or the controller 20 and then to the external device. A chloride concentration can be determined, by the controller 20 and/or the external device 14, based on the signals reflecting the electrochemical reaction. One or more physiological conditions (e.g., electrolyte imbalance, dehydration, cystic fibrosis, etc.) can be determined, by the controller 20 and/or the external device 14, based on at least the determined chloride concentrations. The external device 14 can notify the user of the chloride concentration and/or the one or more physiological conditions with an audible alert, a visual alert, or a tactile alert.

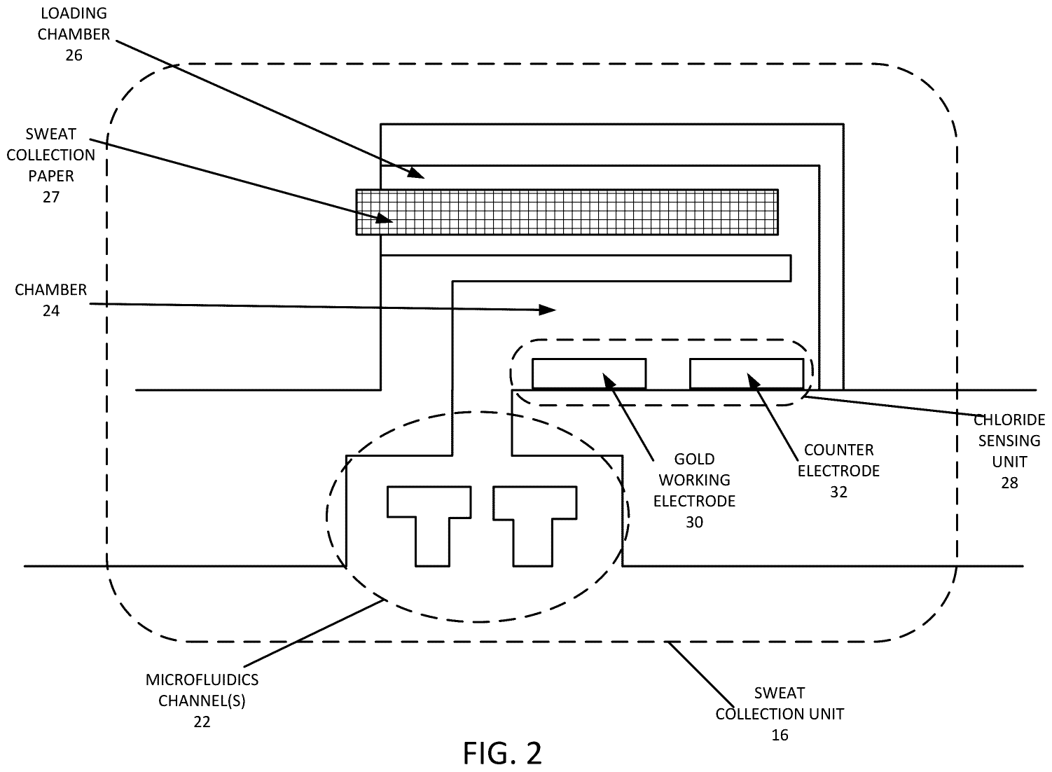

FIG. 2 shows a specific example of the sweat collection unit 16 of the wearable device. The microfluidics channel(s) 22 are positioned so that inlets to the microfluidics channel(s) are near or in contact with the skin of a user wearing the wearable device to attract sweat from the skin using negative capillary pressure. The microfluidics channel(s) 22 direct the sweat into chamber 24 positioned above the microfluidics channel(s). The chamber 24 can hold a given volume of sweat, where the volume the chamber can hold is at least a sufficient amount of sweat to perform the detection of the chloride concentration of the sweat. The chamber 24 is connected to a loading chamber 26 positioned above the chamber to attract older sweat from the chamber so fresh sweat can be continuously collected within chamber 24. The loading chamber 26 can include a sweat collection paper 27 that is disposable and can be inserted into the loading chamber 26 to help attract sweat out of the chamber 24 when the sweat chamber 24 is full and the sweat is in contact with the sweat collection paper. When a sweat collection paper 27 is full of sweat (e.g., cannot attract more sweat) a user can remove the used sweat collection paper and insert a new sweat collection paper 27 in the loading chamber 26. The sweat collection paper 27 may not be paper and instead may be another porous material. The sweat collection paper 27 may include a chemical that reacts with sweat to change the color of the sweat collection paper to notify the user that a given sweat collection paper needs to be removed.

The chamber 24 can also include the chloride sensing unit 28 that can include the gold working electrode 30 positioned a distance from the counter electrode 32. An electrical impedance measurement between the gold working electrode 30 and the counter electrode 32 can be performed (e.g., by signal generator 18 and controller 20 and/or external device 14 (not shown in FIG. 2)) to determine if sufficient sweat is within the chamber and is in contact with both the gold working electrode 30 and the counter electrode 32. When a sufficient amount of sweat is within the chamber 24, then the controller 20 can instruct the signal generator to generate a voltage waveform that can be applied between the gold working electrode 30 and the counter electrode 32. The voltage waveform can be at least one of amperometry, a linear increase square wave voltammetry, a linear step increase voltammetry, a linear ramp increase voltammetry, or a cyclic voltammetry. When the voltage waveform is applied between the gold working electrode 30 and the counter electrode 32 chloride ions in the sweat electrochemically react with the gold molecules of the gold working electrode and signal reflecting the electrochemical reaction of the gold molecules and the chloride ions can be recorded and transmitted to the controller 20 and/or the external device 14 (e.g., by a wired connection or a wireless connection such as Bluetooth® or WIFI). It should be noted that the gold working electrode 30 undergoes electrodissolution when it reacts with the chloride. The area ratio of the gold working electrode 30 and the counter electrode 32 and the distance between the gold working electrode and the counter electrode determine the accuracy, sensitivity, and detection limit of the chloride sensing. A chloride concentration can be determined (by the controller 20 and/or the external device 14) based on the electrochemical signals (e.g., current and/or charge) and one or more physiological conditions can be determined (by the controller 20 and/or the external device 14) based on the chloride concentration. The chloride concentration can be detected over time with multiple applications of the voltage waveform (e.g., timed detections every hour, day, etc., or manually controlled detections based on a user input via external device 14). The controller 20 can change the functionality of the electrodes (e.g., the gold working electrode 30 and the counter electrode 32) depending on the parameters of the electrical signals (e.g., voltage waveforms) configured to be applied through the electrodes.

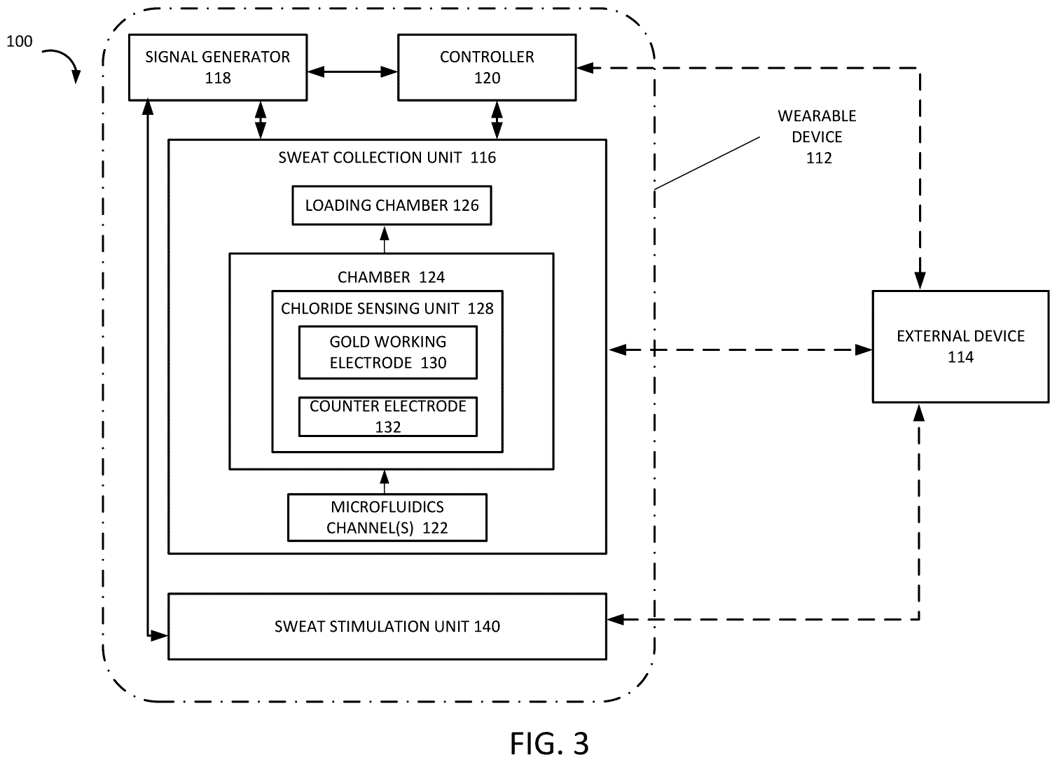

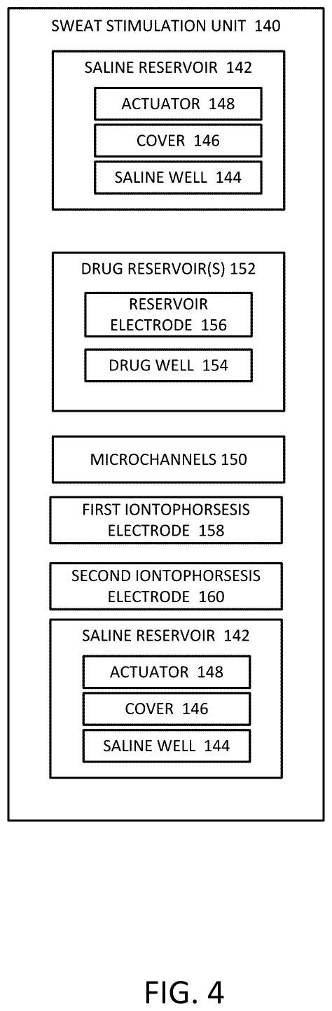

FIG. 3 shows a system 100 that is similar to system 10, but that can also include a sweat stimulation unit 140 in the wearable device 112 that can be in communication with the signal generator 118 and the external device 114. FIG. 4 shows at least some of the components of the sweat stimulation unit 140 in more detail. The sweat stimulation unit 140 can include at least two saline reservoirs 142, each of which can include a saline well 144 that can hold a volume of saline, a cover 146 that can cover at least one opening in the saline well, and an actuator 148 that can break through a wall of the saline well to release the saline from the well when activated (e.g., mechanically, electrically, via a controller, etc.). The sweat stimulation unit 140 can also include microchannels 150 that are in fluid communication with the opening in the saline well (once uncovered) and can direct the released saline towards the skin of the user wearing the wearable device. One or more drug reservoirs 152 can include a drug well 154 that can hold a drug that can trigger a release of sweat (e.g., pilocarpine) and a reservoir electrode 156 that acts at least as a cover on the drug well. The one or more drug reservoirs 152 can also be in fluid communication with one of the microchannels 150 so that when both the saline and the drug are released, they mix within the on microchannel. The sweat stimulation unit 140 can also include a first iontophoresis electrode 158 and a second iontophoresis electrode 160 that can deliver the drug to the user transdermally. For example, the first iontophoresis electrode 158 can act as a cathode and the second iontophoresis electrode 160 can act as an anode for the transdermal drug delivery. However, each iontophoresis electrode may act as either an anode or a cathode depending on the situation, as described below.

The one saline reservoir 142, the drug reservoir 152, and the one connected microchannel 150 can be a drug delivery unit. When an insufficient amount of sweat is in the chamber 124 (of FIG. 3) then the drug can be released from the drug delivery unit to trigger the release of sweat via iontophoresis. The drug can be stored in a repository (e.g., drug reservoir 152) as a dry drug (e.g., a powder or gel) that can mix with saline (e.g., in the connected microchannel 150) after release from the drug delivery unit and before iontophoresis. The saline can be released from storage in the repository before or after the drug is released. A button (e.g., actuator 148) can be configured to release the saline. The button (e.g., actuator 148) may be pressed manually or may be automatically pressed via an instruction from the controller 120. The drug delivery unit can include a positive electrode (e.g., the reservoir electrode 156) that can undergo electrophoresis (e.g., the separation of charged particles by the influence of an electric field that dissolves the electrode) to release the drug. The first iontophoresis electrode 158 can act as the negative electrode for the electrophoresis process. A collection unit (not shown) can include a negative electrode (e.g., the second iontophoresis electrode 160) that can collect the drug from the user's body (with the first iontophoresis electrode 158 now acting as a positive electrode), thereby moving the drug via iontophoresis.

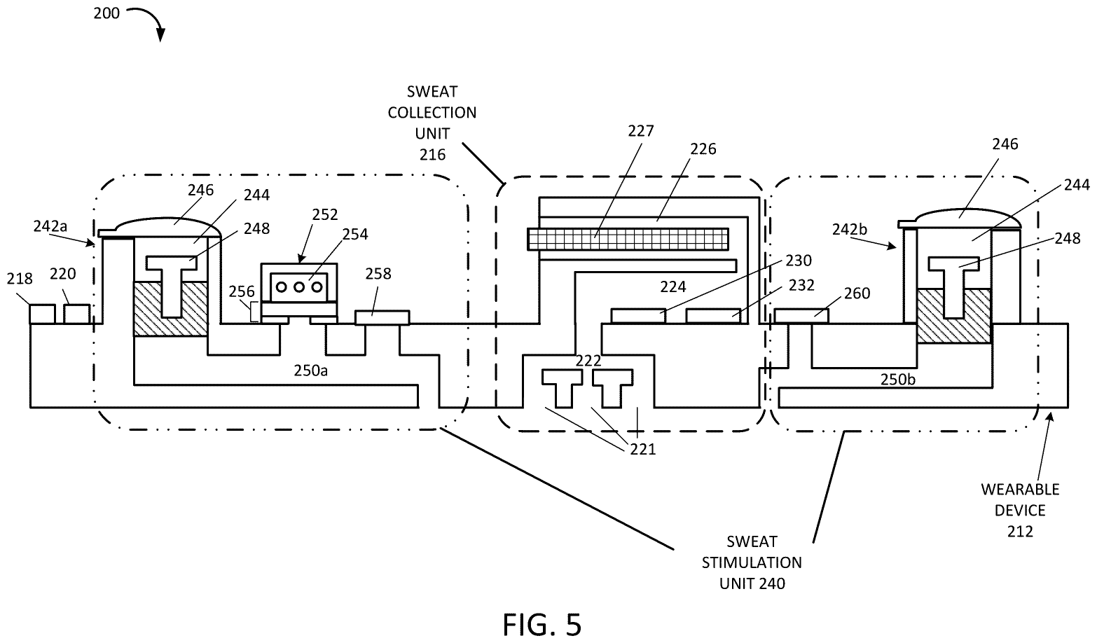

FIG. 5 shows a system 200 (an example implementation of system 100 of FIG. 3), at least a portion of which can be embodied within wearable device 212 that includes both a sweat collection unit 216 and a sweat stimulation unit 240. The wearable device 212 can be secured to the skin of the user (e.g., by an adhesive, a band, etc.) The sweat collection unit 216 can be positioned between two portions of the sweat stimulation unit 240 so that sweat is induced an area beneath the sweat collection unit. The system 200 can include controller 220 that can set parameters for a plurality of voltage waveforms and signal generator 218 coupled to the controller, wired or wirelessly, to generate the plurality of voltage waveforms. The sweat sensing and collection unit (e.g., sweat collection unit 216) can include at least one inlet 221 having microfluidics properties that can collect sweat when positioned on skin of a user by negative capillary pressure attracting the sweat from the skin. A sweat chamber 224 that can hold a volume of the sweat can be connected to the at least one inlet 221 via one or more microfluidics channels 222. A loading chamber 226 can be in fluid communication with the sweat chamber 224 and can hold at least a portion of the volume of sweat from the sweat chamber. The loading chamber 226 can also include a sweat attracting mechanism 227, such as sweat collection paper, that can be configured to continuously draw sweat from the sweat chamber 224 into the loading chamber so that new sweat can continuously enter the sweat chamber. This leads to more accurate measurements that are not muddled by old sweat in the sweat chamber 224. The at least one microfluidics channels 222 can be in fluid communication with the at least one inlet 221, the sweat chamber 224, and the loading chamber 226, so that all are in fluid communication, and can use negative capillary pressure and hydrophilic surfaces to move sweat from the at least one inlet to the sweat chamber and then to the loading chamber.

The sweat chamber 224 can include a gold working electrode 230 and a counter electrode 232 positioned therein. The gold working electrode 230 can be a circular working electrode and the counter electrode 232 can be a semi-circular counter electrode and they can be a distance apart. The area ratio and the distance between the gold working electrode 230 and the counter electrode 232 can be tuned to alter the accuracy, sensitivity, and detection limit of the sensing capabilities. It should be noted that the gold working electrode 230 and the counter electrode 232 have different functionalities based on if the volume/amount of sweat inside the chamber 224 is being measured or if the concentration of chloride in sweat in the chamber is being determined. The gold working electrode 230 and the counter electrode 232 can be used to determine if sufficient sweat is within the chamber 224 and to determine the presence and/or concentration of chloride ions of the sweat within the chamber when there is sufficient sweat. For example, the functionality can depend upon the waveforms/electrical signals applied between the gold working electrode 230 and the counter electrode 232. One of a plurality of voltage waveforms, generated by the signal generator 218, can be delivered between the gold working electrode 230 and the counter electrode 232.

The controller 220 can determine whether a sufficient volume of sweat is within the sweat chamber 224 based on an impedance measured between the gold working electrode 230 and the counter electrode 232 when the one of the plurality of voltage waveforms is delivered therebetween. When a sufficient volume of sweat is within the chamber 224, then another voltage waveform of the plurality of voltage waveforms that causes chloride ions in the sweat to undergo an electrochemical reaction with gold molecules of the gold working electrode 230 can be generated by the signal generator 218 and delivered between the gold working electrode 230 and the counter electrode 232. The voltage waveform can be amperometry, a linear increase square wave voltammetry, a linear step increase voltammetry, a linear ramp increase voltammetry and a cyclic voltammetry. The controller 220 can determine the chloride concentration of the sweat within the sweat chamber 224 based on the electrochemical reaction when signals reflecting the electrochemical reaction are transmitted to the controller. Optionally, the signals reflecting the electrochemical reaction can be transmitted to an external device that includes at least a processor (e.g., a smartphone, a smart watch, a tablet, a computer, etc.) for determining at least the chloride concentration in the sweat. The system 200 can also include a wireless transmitter (not shown) that can transmit the chloride concentration to the external device or a data center for analysis and/or generation of notification to the user based on the analysis.

The system 200 of FIG. 5 can also include the sweat stimulation unit 240 that can induce a user to sweat if the user is not sweating enough. The sweat stimulation unit 240 can include two saline reservoirs 242 a, b, a first saline reservoir 242 a configured on a side of the sweat sensing and collection unit 216 and a second saline reservoir 242 b configured on an opposite side of the sweat sensing and collection unit. Each of the saline reservoirs 242 a, b include a well 244 that can hold and store saline, a mechanical actuator 248 that can open a portion of the well holding the saline when actuated by the user, and a cover 246 over the mechanical actuator that has to be removed before the mechanical actuator can be actuated by the user. Microchannels 250 a, b are in fluid communication with the saline reservoirs once the mechanical actuator has opened a portion of the saline wells and can hold and guide the saline towards the skin of the user. A first microchannel 250 a is in communication with the first saline reservoir 242 and the skin of the user and a second microchannel 250 b is in communication with the second saline reservoir and the skin of the user. A drug reservoir 252 is positioned between the first saline reservoir 242 a and the sweat sensing and collection unit 216. The drug reservoir 252 can include a well 254 that can hold and store a drug that can induce sweating and can have an opening to the first microchannel 250 a. The drug reservoir 252 can also include a reservoir electrode 256 comprising a circular gold film above an annulus electrode that is inert under 2V. The circular gold film of the reservoir electrode 256 can cover the opening to the well 254 and electrodissolve to release the drug into the saline in the first microchannel 250 a. The annulus electrode can be any material that is dielectric or electrically conductive and inert (e.g., not electrochemically active) under 2V, such as platinum, silver chloride, or aluminum oxide. The drug can be, for example, pilocarpine and can, for example, be stored in a solid form as a powder that can be combined with saline (e.g., in the first microchannel 250 a) so that it can be delivered via iontophoresis.

The sweat stimulation unit 240 also includes a first iontophoresis electrode 258 and a second iontophoresis electrode 260. The first iontophoresis electrode 258 is positioned between the drug reservoir 252 and the sweat sensing and collection unit 216 and near an opening from the first microchannel 250 a to the skin. The first iontophoresis electrode 258 can be a half-moon shape and can be any material that is electrically conductive and inert (e.g., not electrochemically active) under 1V, such as, gold, platinum, or silver chloride. The second iontophoresis electrode 260 is positioned between the second saline reservoir 242 and the sweat sensing and collection unit and near an opening from the second microchannel 250 b to the skin. The signal generator 218 can generate another of the plurality of voltage waveforms that can be applied from the reservoir electrode 256 (e.g., from the circular gold film of the reservoir electrode) to the first iontophoresis electrode 258 to electrodissolve the circular gold film of the reservoir electrode and release the drug into the first microchannel 250 a. The another of the plurality of voltage waveforms applied between the reservoir electrode 256 and the first iontophoresis electrode 258 can have a constant voltage between 1.2 V and 1.8V with the reservoir electrode being positive and the first iontophoresis electrode being negative.

Once the drug and the saline are both released into the first microchannel 250 a, then yet another of the plurality of voltage waveforms can be generated by the signal generator 218 and applied between the annulus electrode of the reservoir electrode 256 and the first iontophoresis electrode 258 to iontophoretically collect the drug near the first iontophoresis electrode. The yet another voltage waveform can have a constant voltage less than 1 V with the reservoir electrode 256 being positive and the first iontophoresis electrode 258 being negative. When still another voltage waveform is generated by the signal generator 218 and applied from the first iontophoresis electrode 258 to the second iontophoresis electrode 260, then the drug can be iontophoretically delivered into the skin of the user where the drug induces sweating. The still another of the plurality of voltage waveforms applied from the first iontophoresis electrode 258 to the second iontophoresis electrode 260 can have a constant voltage less than 1 V, with the first iontophoresis electrode being positive and the second iontophoresis electrode being negative. The sweat sensing and collection unit 216 can then determine a chloride concentration of the sweat as described above. The determined chloride concentration can be used, for example by the external device, to determine one or more physiological conditions of the user, such as cystic fibrosis, dehydration, or an electrolyte imbalance.

The controller 220 of system 200 may operate in a manual, semi-autonomous, or autonomous fashion in detecting if the sweat level is sufficient or not; if the on-skin sweat is insufficient, the sweat can be stimulated. The controller 220 can determine whether the sufficient volume of sweat within the sweat chamber 224 based on the electrical impedance measured between the gold working electrode 230 and the counter electrode 232. If the volume of sweat within the sweat chamber 224 is insufficient, then the controller 220 can send a notification to a user, via an external device (e.g., a mobile device such as a smart phone or smart watch, a tablet, or a computer) to trigger the sweat stimulation unit 240. Optionally, the controller 220 may automatically trigger the sweat stimulation unit 240 without the user's input. If the volume of sweat within the sweat chamber 224 is sufficient, the controller 220 can then determine the chloride concentration of the sweat based on signals indicative of the electrochemical reaction of the gold molecules with the chloride ions when the other voltage waveform that causes chloride ions in the sweat to undergo an electrochemical reaction with gold molecules of the gold working electrode is applied.

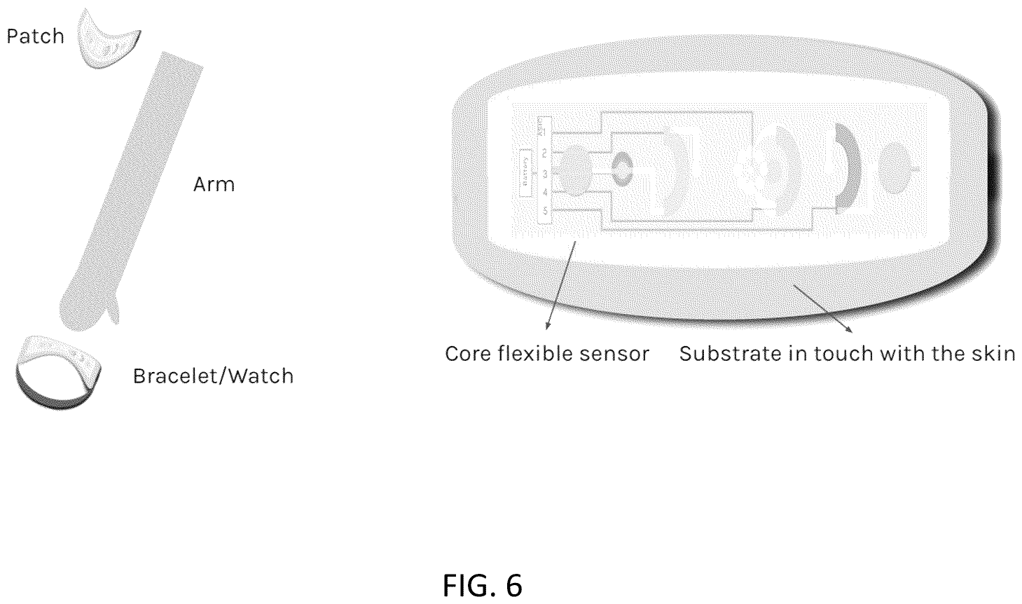

FIG. 6 shows examples of the wearable device. The wearable device can be a patch or an arm band such as a bracelet or watch or a sweat band. If the wearable device is a patch, it can include an adhesive that can keep the underside of the wearable device in contact and/or near the skin of a user. If the wearable device is an arm band it can include a band or another securing means to keep it near and/or in contact with the skin of the user. The wearable device can include a core flexible sensor, that can include any or all of the wearable device components described above in their various configurations, and a substrate that can be in contact with the skin and can include the adhesive, band, or other securing means.

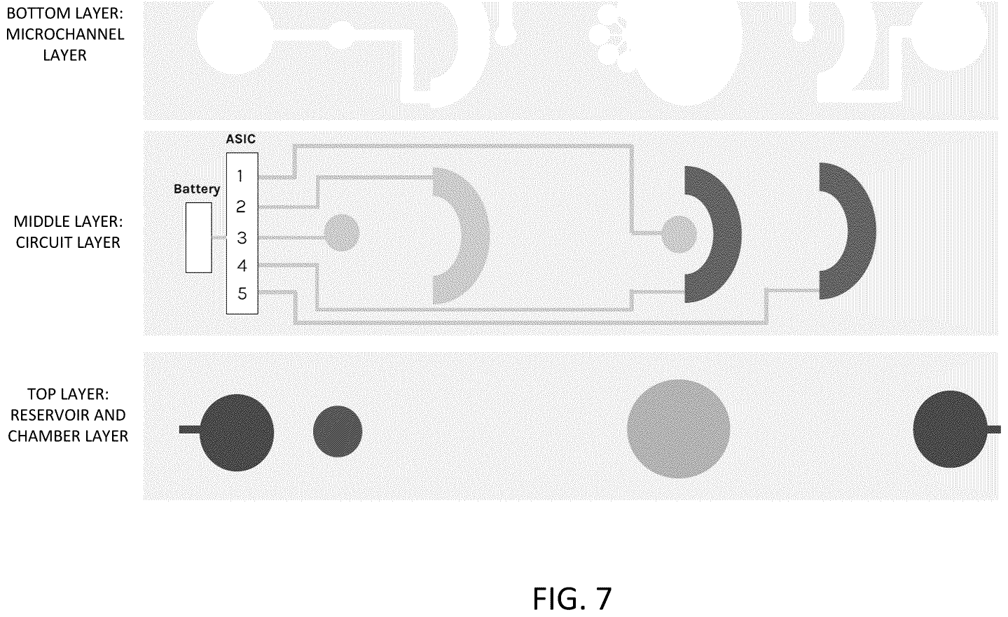

FIG. 7 shows bottom views of the different layers of an example embodiment of the core flexible sensor of FIG. 6. The bottom layer of the core flexible sensor can include the microchannel and microfluidics channels that can fluidly connect the different components of the sweat stimulation and the sweat collection unit to the skin. The microchannels and/or microfluidics channels can move the saline and the drug towards the skin and collect sweat from the skin, depending on if they are part of the sweat stimulation unit or the sweat collection unknit. The middle layer includes the circuitry including all the electrodes, the electrical connections, the battery (e.g., a signal generator), and the ASIC (e.g., the controller). The top layer can include the saline and drug reservoirs and the chambers for collecting sweat and loading the sweat collection paper.

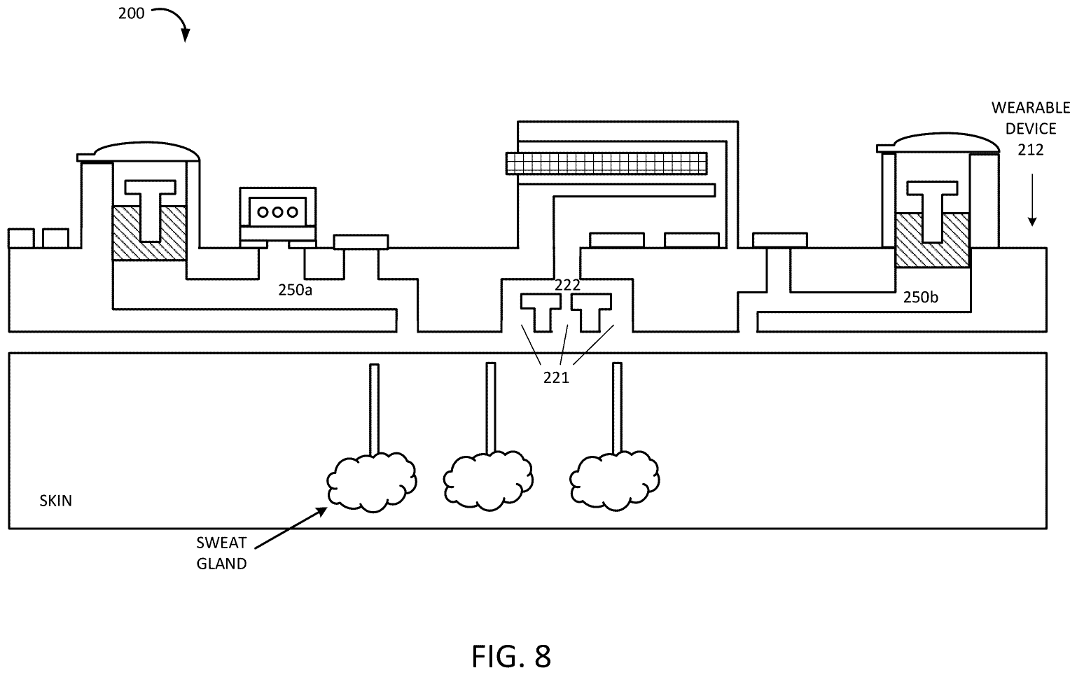

FIGS. 8-15 illustrate an example operation of the system 200 shown in FIG. 5 and described in detail above. FIG. 8 shows the system 200 as a wearable device 212 being positioned near and/or in contact with the skin of the user. The wearable device 212 can be a patch that can include an adhesive that can secure the wearable device to the skin. The wearable device 212 can also be an armband or a wristband that can be secured to the skin with a band or an elastic. The wearable device 212 is positioned on the skin such that the inlets 221 (of microfluidics channels 222) and microchannels 250 a, b are in fluid communication with the skin. The wearable device 212 can be positioned by the user wearing the device or by an additional user that can be there to help the user wearing the wearable device work the systems described herein (e.g., when the user wearing the device is too young, too old, or too infirm to work the systems on their own). Once the wearable device 212 is positioned on the skin of the user then the wearable device can be used.

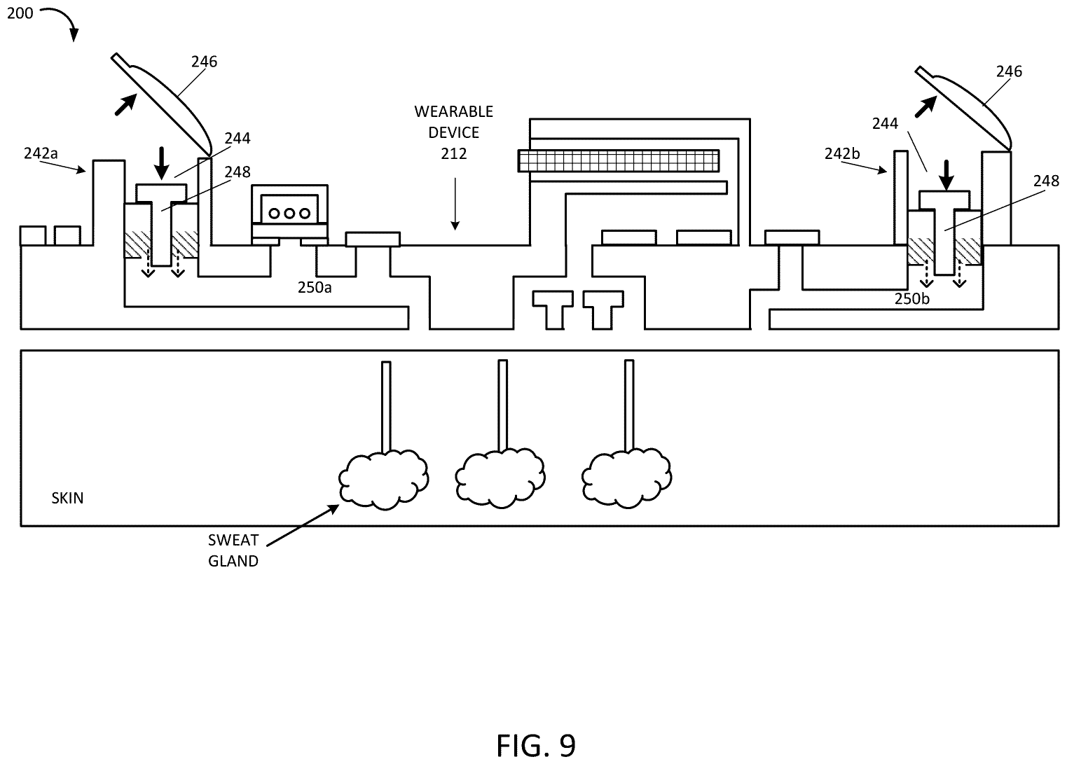

FIG. 9 shows the covers 246 of both the saline reservoirs 242 a, b being removed (e.g., partially or entirely) by a user (either the wearer or another user assisting the wearer) and then the mechanical actuators 248 of the saline reservoirs being actuated. The mechanical actuators 248 are shown as being pressed down to rupture a lower portion of the saline well 244 of the saline reservoirs 242 a, b, it is understood that this is only one example of how a mechanical actuator can be actuated. When the bottom of the saline well 244 is ruptured then the saline held in the saline reservoirs 242 a, b is released into the now connected microchannels 250 a, b where it can flow towards the skin of the user wearing the wearable device 212 to fill the microchannels. For example, all of the surfaces inside the microchannel 250 a, b can be hydrophilic. When the saline is in contact with the hydrophilic microchannel surface then the surface contact angle is less than 90°, which generates a concave liquid-air interface and a negative capillary pressure which forces the liquid front of the saline to move towards the outlet of the microchannel 250 a, b that the liquid is in contact with and/or near the skin of the user.

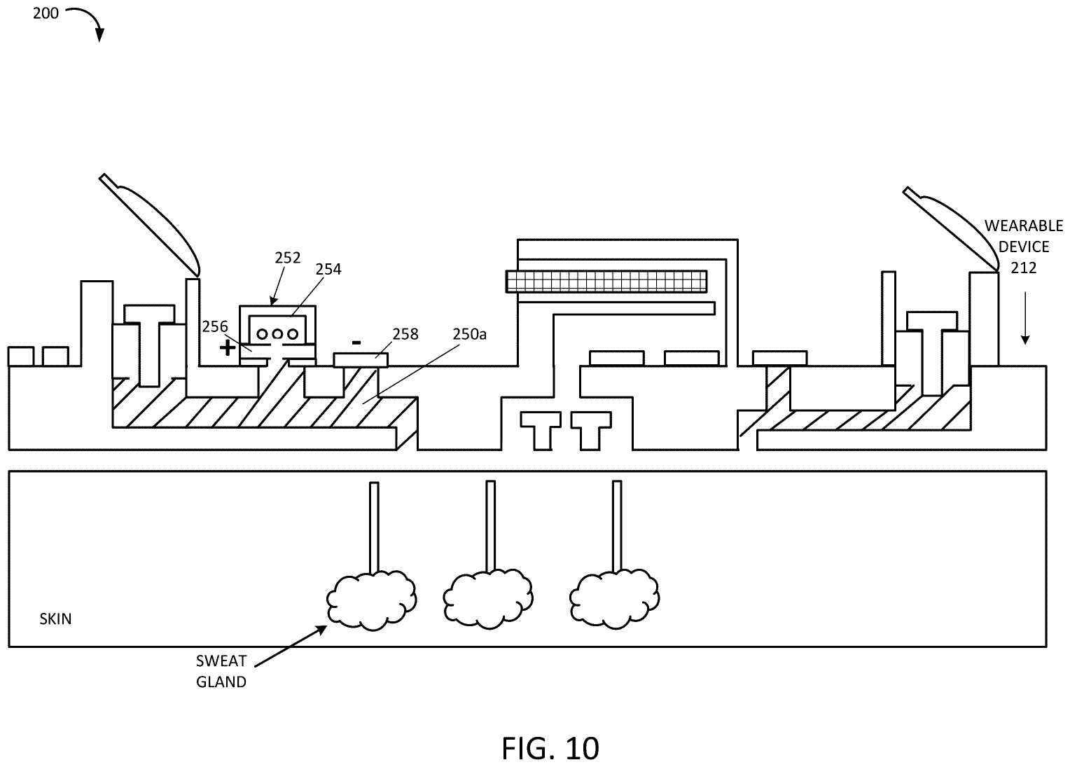

FIG. 10 shows the wearable device 212 with the saline held in the microchannels 250a, b and the drug stored in the drug reservoir 252 being released. The drug can be, for example, a solid form of pilocarpine such as a powder that can mix with the saline when released. Utilizing dry storage of pilocarpine greatly increases its shelf life compared to traditional gelled pilocarpine because drug degradation is less likely. The drug reservoir 252 includes the well 254 storing the drug and a drug reservoir electrode 256 between the drug well and an opening to the first microchannel 250 a.

The drug reservoir electrode 256 includes a gold film electrode and an annulus electrode. The annulus electrode can be any material that is dielectric or electrically conductive and inert (e.g., not electrochemically active) under 2V (e.g., platinum, silver chloride, aluminum oxide, or the like). A voltage waveform is applied from a portion of the drug reservoir electrode 256, acting as positive electrode (+), to a first iontophoresis electrode 258, acting as the negative electrode for a time. The voltage waveform is a constant voltage between 1.2 V and 1.8 V. The first iontophoresis electrode 258 can have a half-moon shape and can be any material that is electrically conductive and inert (e.g., not electrochemically active) under 1V (e.g., gold, platinum, silver chloride, or the like). The voltage waveform is applied for a time to dissolve the gold film electrode portion of the reservoir electrode 256 to release the drug into the first microchannel 250a.

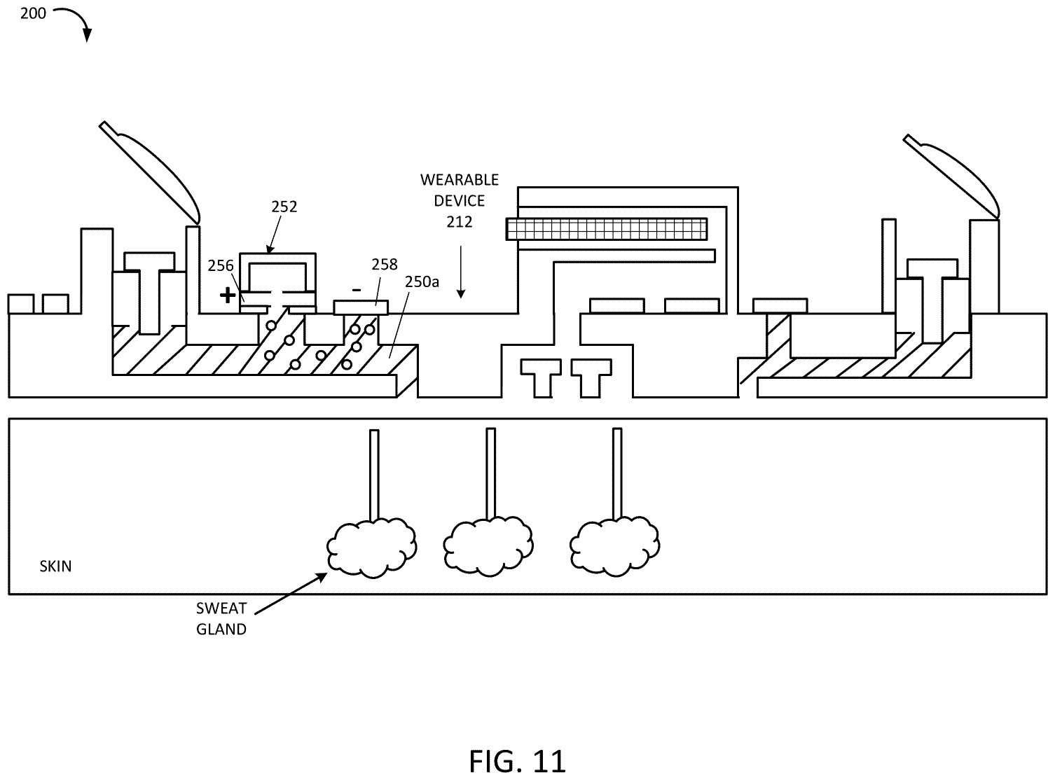

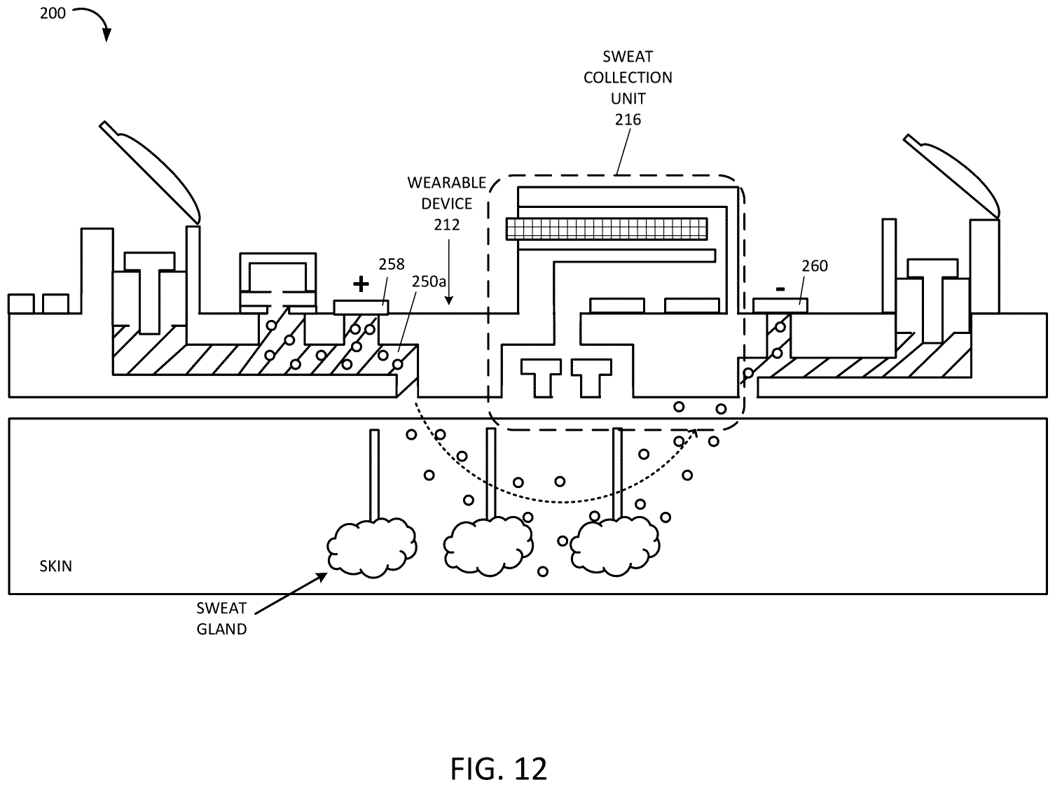

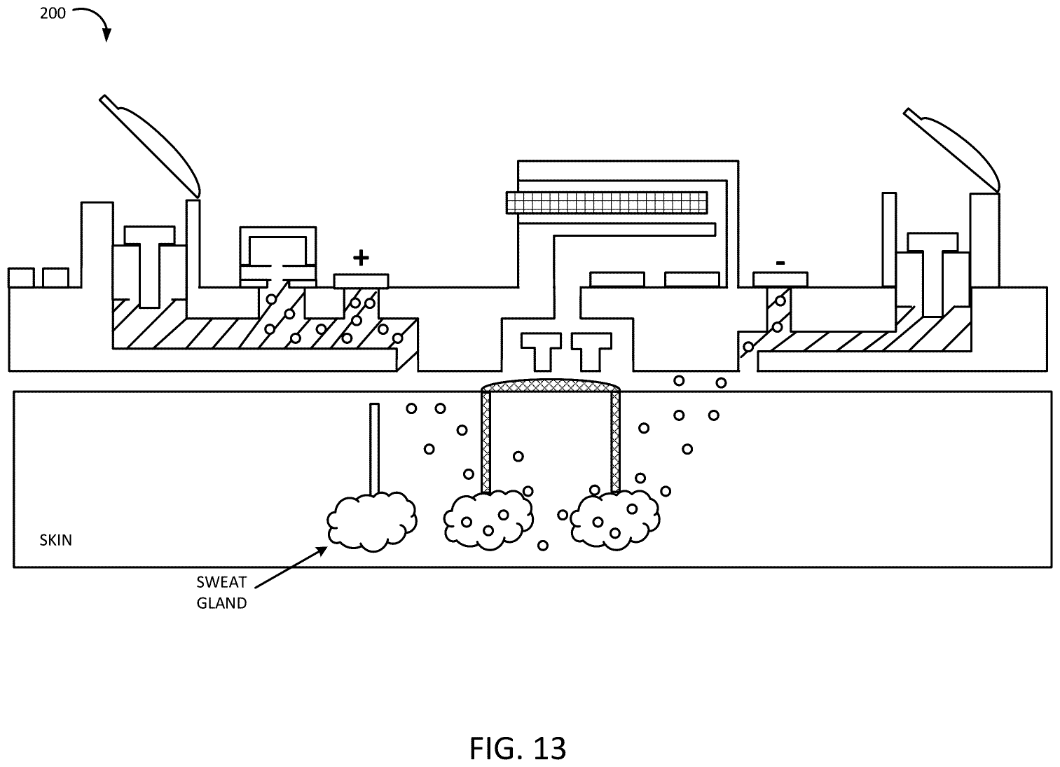

As shown in FIG. 11, once the drug is released and diffuses out of the drug reservoir 252 and mixes with the saline in the first microchannel 250 a, then another voltage waveform is applied between the annulus electrode of the reservoir electrode 256, as the positive electrode, and the first iontophoresis electrode 258, as the negative electrode. This voltage waveform can have a constant voltage less than or equal to 1 V. Applying this voltage collects the released drug in the first microchannel 258 near the first iontophoresis electrode 258. FIG. 12 shows the other voltage (e.g., a constant voltage less than or equal to 1 V) being applied between the first iontophoresis electrode 258, acting as the positive electrode, and the second iontophoresis electrode 260, acting as the negative electrode and positioned on the other side of the sweat sensing and collection unit 216. This voltage is applied to iontophoretically deliver the drug into the skin and to the sweat glands in the skin. The second iontophoresis electrode 260 can be any material that is dielectric or electrically conductive and inert (e.g., not electrochemically active) under 1 V (e.g., platinum, gold, silver chloride, aluminum oxide, or the like). FIG. 13 shows the sweat being generated by the sweat glands in the user's skin in response to the iontophoretically delivered drug.

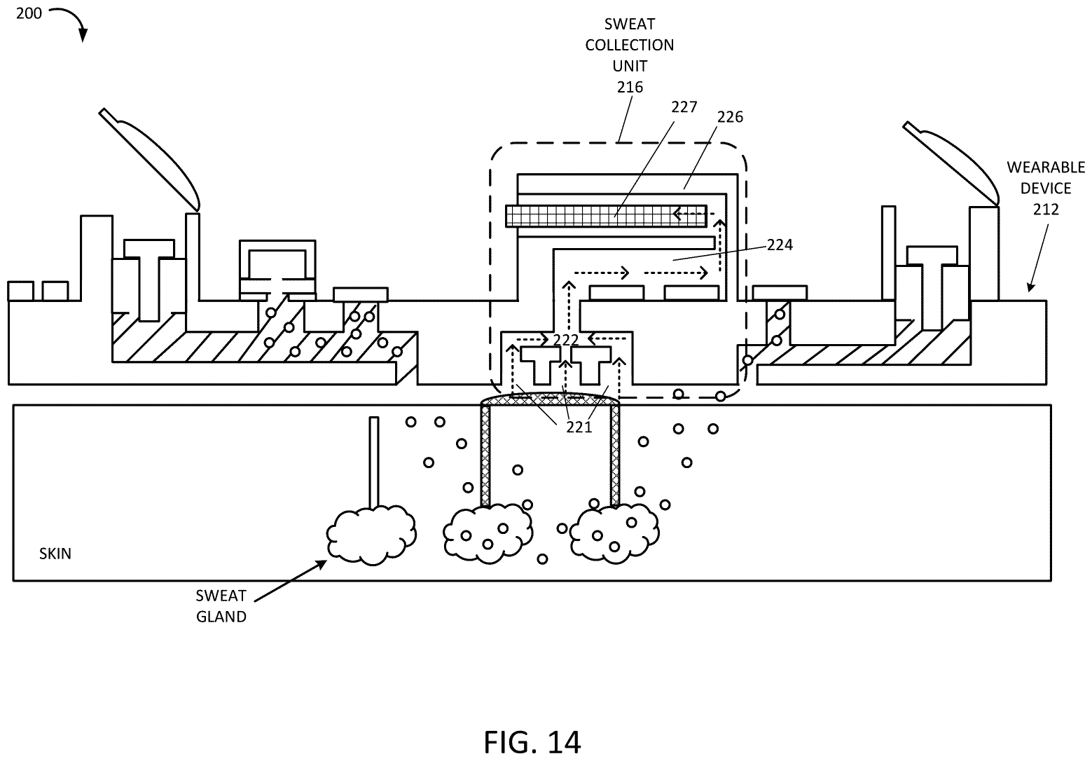

FIG. 14 shows the sweat filling the microfluidic channel(s) of the sweat sensing and collection unit 216 after transdermal application of the sweat inducing drug (e.g., pilocarpine) via the sweat stimulation unit. In another embodiment not shown, the sweat stimulation unit may not be activated, and the user may be sweating on the user's own. When the wearable device 212 is near and/or in contact with the sweat on the skin of the user, then negative capillary pressure attracts the sweat into the microfluidics channel(s) 222 via inlets 221 and moves the sweat to the chamber 224 (e.g., sweat chamber) for chloride analysis. A disposable sweat collection paper 228 can be loaded into the loading chamber 226 adjacent to and/or in communication with the chamber 224 (e.g., sweat chamber) via a microfluidic channel 222 to help attract sweat to continue out of the chamber after the chamber is full. The disposable sweat collection paper 228 can be porous to enhance the liquid attraction capability and can be replaced. Attracting the sweat out of the chamber 224 (e.g., sweat chamber) and into the loading chamber 226 and/or disposable sweat collection paper 227 allows fresh sweat to replace the old sweat in the chamber for better chloride concentration analysis.

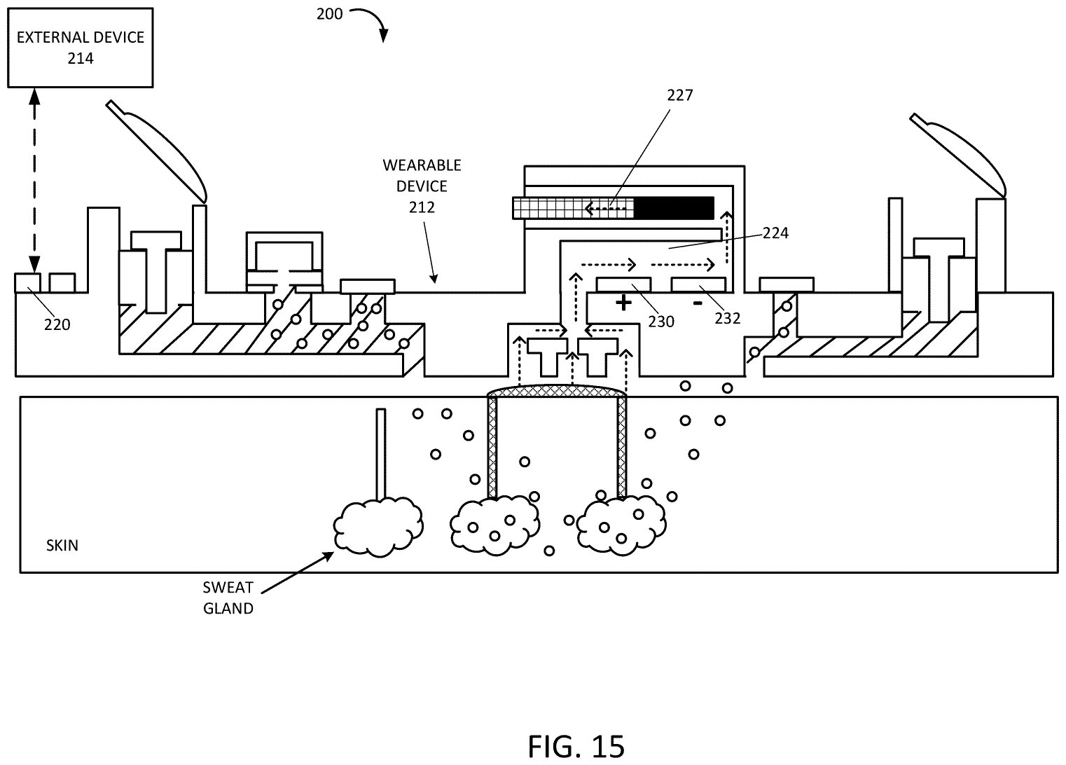

FIG. 15 shows the chamber 224 (e.g., sweat chamber) once it has filled and the disposable sweat collection paper 227 having partially filled with sweat as well to show the chamber is full. FIG. 15 also shows a voltage waveform being applied from the gold working electrode 230 (e.g., circular) and the counter electrode 232 (e.g., semi-circular and can be gold, silver chloride, platinum, or the like) within the chamber 224 (e.g., sweat chamber) to determine the presence and concentration of chloride ions in the sweat. The chloride ion concentration is determined based on electrochemical signals (current or charge) when the chloride ions in the sweat react with gold molecules of the gold working electrode 230 as the voltage waveform is being applied. The voltage waveform can be at least one of amperometry, a linear increase square wave voltammetry, a linear step increase, voltammetry, a linear ramp increase voltammetry, or a cyclic voltammetry. FIG. 15 also shows the wearable device 212 in communication with an external device 214 (via a controller 220 and a wireless transceiver not shown) that can notify the user of the of the determined chloride concentration. The external device can also analyze the chloride concentration and notify the user of one or more physiologic conditions based on the chloride concentration. All voltage waveforms of FIGS. 8-15 are generated by signal generator 218 with parameters configured by controller 220 (as marked in FIG. 5).

### IV. Method

Another aspect of the present disclosure can include methods that can be used to evaluate and/or monitor one or more physiological conditions of user by determining a chloride concentration in the user's sweat. The methods can be implemented via a wearable device (as described in FIGS. 1-7 and an example operation shown in FIGS. 8-15) that can include a sweat collection unit and, optionally, a sweat stimulation unit and an external device (e.g., a smartphone, smart watch, tablet, computer, etc.) in communication with the wearable device.

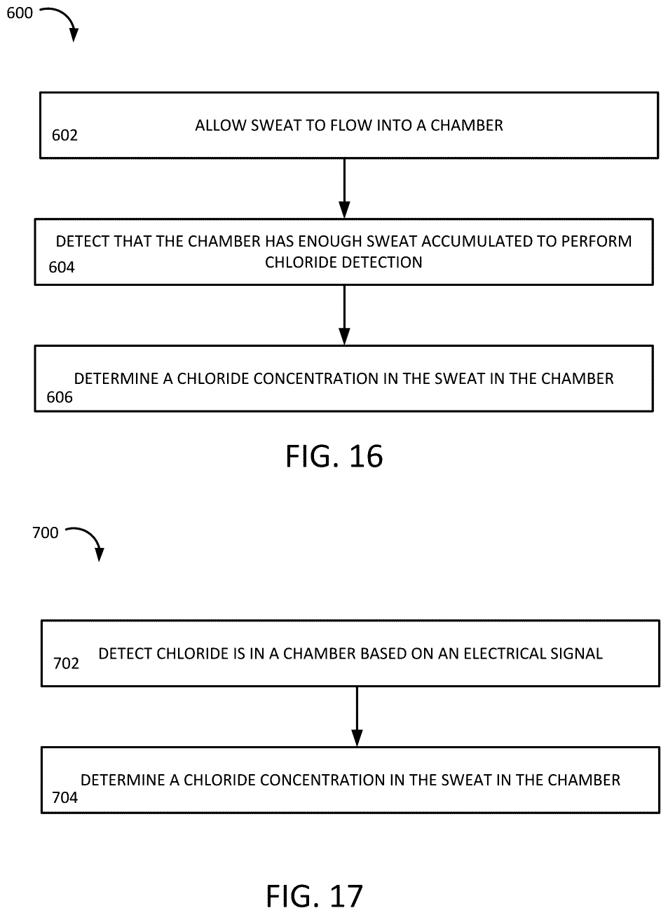

Methods 600-900 (FIGS. 16-19) are illustrated as process flow diagrams with flowchart illustrations that can be implemented by one or more components of the systems shown in FIGS. 1-5. For purposes of simplicity, the methods 600-900 are shown and described as being executed serially; however, it is to be understood and appreciated that the present disclosure is not limited by the illustrated order as some steps could occur in different orders and/or concurrently with other steps shown and described herein. Moreover, not all illustrated aspects may be required to implement the methods 600-900.

FIG. 16 illustrates a method 600 for determining a chloride concentration in the sweat of a user. At step 602, sweat of the user is allowed to flow into a chamber (e.g., of a sweat sensing and collection unit). Sweat is allowed to flow into the chamber when the wearable device including the sweat sensing and collection unit is positioned on and/or near the skin of the user. At step 604, a processor can detect that the chamber has accumulated enough sweat to perform chloride detection. Detecting that the chamber has accumulated enough sweat to perform chloride detection can include determining, by the processor, whether an amount of sweat fills the chamber of a sweat sensing and collection unit, based on an impedance measured between a gold working electrode and a counter electrode of a chloride sensing unit located within the chamber. When the impedance indicates that a sufficient amount of sweat fills the chamber, then at step 606 a chloride concentration of the sweat in the chamber can be determined. The chloride concentration of the sweat in the chamber can be determined by delivering a voltage waveform between the gold working electrode and the counter electrode within the chamber and measuring signals associated with an electrochemical reaction between the chloride ions in the sweat and the gold molecules of the gold working electrode. The voltage waveform can be one of amperometry, a linear increase square wave voltammetry, a linear step increase voltammetry, a linear ramp increase voltammetry, and a cyclic voltammetry. When the voltage waveform is delivered molecules from the gold working electrode electrochemically react to the chloride ions of the sweat in the chamber and signals (e.g., current and/or charge) can be measured.

FIG. 17 illustrates a method 700 for determining a chloride concentration in sweat. At step 702, chloride in the chamber can be detected based on an electrical signal. The electrical signal can be applied between the gold working electrode and the counter electrode in the chamber of the sweat sensing and collection unit. The electrical signal can be one of amperometry, a linear increase square wave voltammetry, a linear step increase voltammetry, a linear ramp increase voltammetry, and a cyclic voltammetry. Moreover, at step 704 the chloride concentration of the sweat in the chamber can be determined with application of the same electrical signal. When the electrical signal is delivered between the gold working electrode and the counter electrode, then molecules from the gold working electrode electrochemically react to the chloride ions of the sweat in the chamber and signals (e.g., current and/or charge) can be measured to determine the chloride concentration. Information regarding the chloride concentration determined in methods 600 and 700 can be used to determine one or more physiological conditions of the user based on the chloride concentration of the sweat in the chamber. The chloride concentrations can be detected once, once a day, once an hour, or once a half-hour, for example, based on the physiological conditions being tested for.

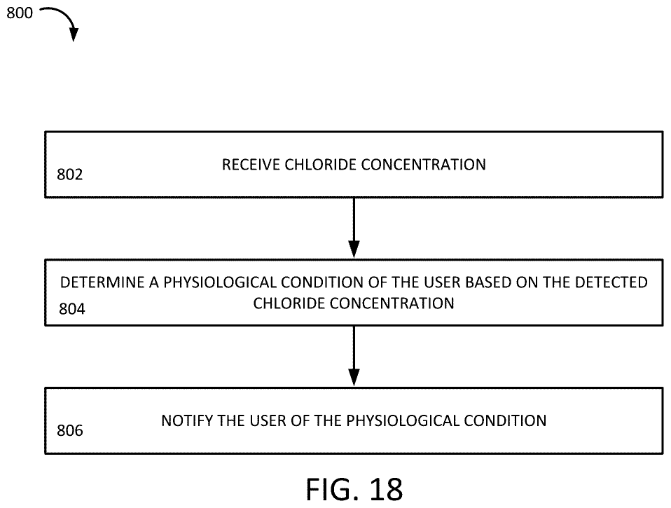

FIG. 18 illustrates a method 800 that a processor of an external device and/or a built-in controller of the wearable device can perform to notify a user of a physiological condition related to chloride concentration in the user's sweat. At step 802, the external device can receive the chloride concentration (that can be determined based on methods 600 and/or 700). At step 804, a physiological condition of the user can be determined based on the detected chloride concentration in the sweat. The determination can be based, for example, on comparing the detected chloride concentration with known chloride concentrations indicative of various physiological conditions. The physiological condition can be, for example, one of dehydration, electrolyte imbalance, or cystic fibrosis. The condition being determined may be previously identified and the determination can be a yes or no determination based on if the chloride concentration detected meets one or more criteria for that physiological condition. For example, if the wearable device is being used to test for cystic fibrosis, and the processor is instructed of this, then the processor will not determine if the user is dehydrated or not, and vice versa. At step 806, the user can be notified of the physiological condition. The notification can be at least one of visual, tactile, or audible. The notification may come from the wearable device or an external device comprising at least the processor and a notification device (e.g., display, vibrator, LED, speaker, etc.).

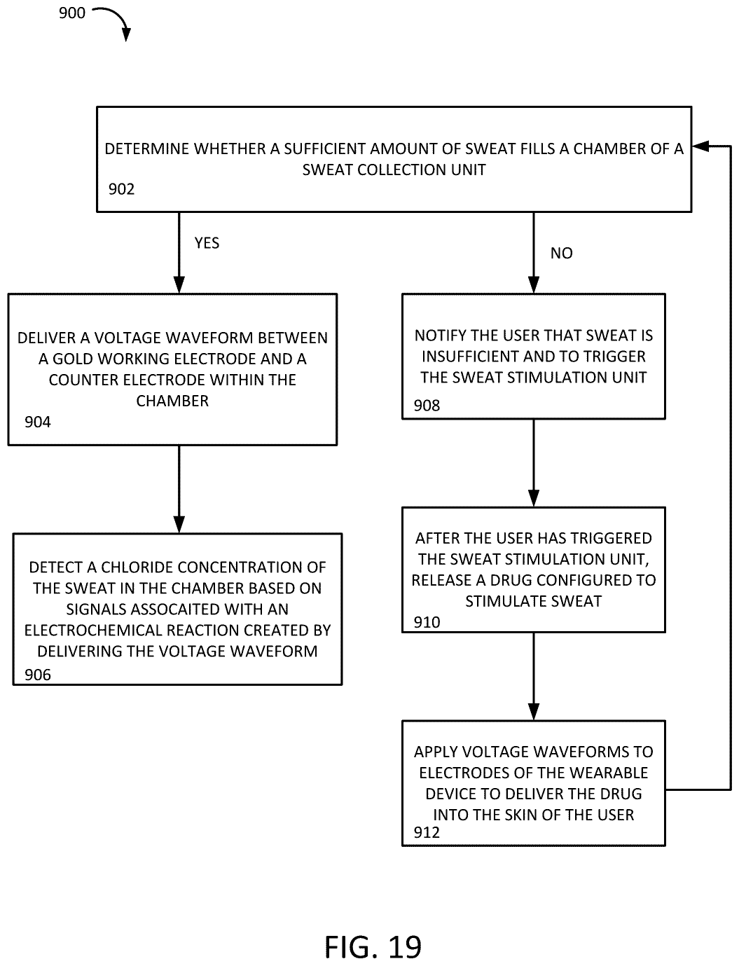

FIG. 19 shows a method 900 for determining if only the sweat sensing and collection unit needs to be active or if a sweat stimulation unit needs to be activated before the sweat sensing and collection unit can determine a chloride concentration in a user's sweat. At step 902, a determination can be made as to whether a sufficient amount of sweat fills a chamber of the sweat sensing and collection unit. The determination can be based on an impedance measurement between a gold working electrode and a counter electrode positioned within the chamber of the sweat sensing and collection unit that fills with sweat. If sufficient sweat is determined to be within the chamber, then at step 904, like described in methods 600 and 700, a voltage waveform can be delivered between the gold working electrode and the counter electrode within the chamber (it should be noted that the electrodes can have at least two different functionalities). The voltage waveform can cause an electrochemical reaction between gold molecules of the gold working electrode and chloride ions in sweat. Signals (e.g., charge and/or current) associated with the electrochemical reaction can be measured. At step 906, the chloride concentration of the sweat in the chamber is detected based on the signals associated with the electrochemical reaction created by delivering the voltage waveform.

If sufficient sweat (e.g., a sufficient volume of sweat) is not determined to be within the chamber at step 902, then at step 908 the user can be notified that the sweat is insufficient and that the user must trigger the sweat stimulation unit of the system. The user can physically trigger the sweat stimulation unit (e.g., depress an actuator/button). Optionally, the system may automatically trigger the sweat stimulation unit with another instruction from a controller of the system in communication with the actuator/button. At step 910, after the user has triggered the sweat stimulation unit, a drug configured to stimulate sweat (e.g., pilocarpine or the like) can be released from a drug storage reservoir of the sweat stimulation unit via electroporation of an electrode covering an opening of the drug storage reservoir. At step 912, voltage waveforms can be applied to electrodes of the wearable device to deliver the drug into the skin of the user to induce sweating. The drug can be delivered through a saline pathway in microchannel of the wearable device that is released when the user triggers the sweat stimulation unit. The drug is delivered through the saline pathway, into the skin of the user via iontophoresis between an electrode at least partially covering the opening of the drug storage reservoir, a first iontophoresis electrode, and a second iontophoresis electrode. Step 902 can then repeat to determine if after sweat has been induced there is a sufficient amount/volume of sweat in the chamber to determine the chloride concentration. One or more cycles of the sweat stimulation may be needed during the useful life of the wearable device so the wearable device can include a plurality of drug storage reservoirs and the method 900 can also include selecting one of the drug storage reservoirs to release the drug from to induce sweat.

From the above description, those skilled in the art will perceive improvements, changes and modifications. Such improvements, changes and modifications are within the skill of one in the art and are intended to be covered by the appended claims. All patents, patent applications, and publications cited herein are incorporated by reference in their entirety.

What is claimed is:

1. A wearable device comprising:
a sweat collection unit comprising:
at least one microfluidics channel with hydrophilic surfaces configured to collect sweat from a user's skin,
a chamber configured to hold the sweat, and
a loading chamber comprising disposable sweat collection paper, wherein the disposable sweat collection paper is configured to attract the sweat so the chamber is continuously filling with fresh sweat;
a chloride sensing unit located within the chamber comprising a gold working electrode and a counter electrode, wherein the gold working electrode and the counter electrode are configured to:
determine whether the sweat is within the chamber, and
when the sweat is within the chamber, deliver a voltage waveform that causes chloride ions in the sweat to undergo an electrochemical reaction with gold molecules of the gold working electrode,
wherein signals reflecting the electrochemical reaction between the chloride ions and the gold molecules are transmitted to an external device, and
wherein the wearable device is flexible.

2. The wearable device of claim 1, further comprising a drug delivery unit comprising a drug configured to trigger a release of sweat,
wherein when the sweat is not available inside the chamber, the drug is released from the drug delivery unit to trigger the release of sweat via iontophoresis.

3. The wearable device of claim 2, wherein the drug delivery unit comprises a positive electrode configured to undergo electrophoresis to release the drug and a collection unit comprises a negative electrode to collect the drug.

4. The wearable device of claim 2, wherein the released drug is collected from the user's body via iontophoresis.

5. The wearable device of claim 2, wherein the drug is stored in a repository as a dry drug that is mixed with saline after release from the drug delivery unit and before the iontophoresis.

6. The wearable device of claim 5, further comprising a button configured to release the saline from storage before and/or as the drug is released.

7. The wearable device of claim 1, wherein impedance is measured between the gold working electrode and the counter electrode to determine whether the sweat is within the chamber.

8. The wearable device of claim 1, wherein the gold working electrode is a circular working electrode and the counter electrode is a semi-circular counter electrode.

9. The wearable device of claim 1, wherein the counter electrode comprises gold, silver chloride, and/or platinum.

10. A system comprising:
a controller configured to set parameters for a plurality of voltage waveforms;
a signal generator, coupled to the controller, configured to generate the plurality of voltage waveforms; and
a sweat sensing and collection unit comprising:
at least one inlet having microfluidic properties and configured to collect sweat when positioned on skin of a user by negative capillary pressure attracting the sweat from the skin,
a sweat chamber configured to hold a volume of the sweat and comprising:
a gold working electrode, and
a counter electrode,
wherein the gold working electrode and the counter electrode are configured to:
deliver one of the plurality of voltage waveforms therebetween, wherein the controller determines whether a sufficient volume of sweat is within the sweat chamber based on an impedance measured between the gold working electrode and the counter electrode;
when the sufficient volume of sweat is within the chamber, deliver another voltage waveform of the plurality of voltage waveforms that causes chloride ions in the sweat to undergo an electrochemical reaction with gold molecules of the gold working electrode, wherein the controller determines the chloride concentration of the sweat within the chamber based on the electrochemical reaction;
a loading chamber in fluid communication with the sweat chamber and configured to hold at least a portion of the volume of sweat, wherein the loading chamber includes a sweat attracting mechanism configured to continuously draw sweat from the sweat chamber into the loading chamber; and
at least one microchannel in fluid communication with the at least one inlet, the sweat chamber, and the loading chamber and configured to use negative capillary pressure to move sweat from the at least one inlet to the sweat chamber and to the loading chamber.

11. The system of claim 10, further comprising a wireless transmitter configured to transmit the chloride concentration to a mobile device or data center for analysis and/or generation of notifications to the user based on the analysis

12. The system of claim 10, wherein at least a portion of the system is embodied within a wearable device that is configured to be secured to the skin of the user.

13. The system of claim 10, wherein the system further comprises a sweat stimulation unit comprising:
two saline reservoirs, a first saline reservoir configured on a side of the sweat sensing and collection unit and a second saline reservoir configured on an opposite side of the sweat sensing and collection unit, wherein each of the saline reservoirs comprises:
a well configured to hold saline;
a mechanical actuator configured to open a portion of the well holding the saline when actuated by the user;
a cover over the mechanical actuator configured to be removed before the mechanical actuator can be actuated by the user;
a first microchannel in communication with the first saline reservoir and the skin of the user and configured to hold and guide the saline towards the skin;
a second microchannel in communication the second saline reservoir and the skin of the user and configured to hold and guide the saline towards the skin;
a drug reservoir positioned between the first saline reservoir and the sweat sensing and collection unit, the drug reservoir comprising:
a well configured to hold a drug and having an opening to the first microchannel, and
a reservoir electrode comprising a circular gold film above an annulus electrode that is inert under 2V, wherein the circular gold film is configured to cover the opening to the well and electrodissolve to release the drug into the saline in the first microchannel;
a first iontophoresis electrode positioned between the drug reservoir and the sweat sensing and collection unit and near an opening from the first microchannel to the skin, wherein the circular gold film of the reservoir electrode electrodissolves when another of the plurality of voltage waveforms is applied from the circular gold film to the first iontophoresis electrode and the drug is released into the first microchannel, wherein the drug is iontophoretically collected in the first microchannel near the first iontophoresis electrode when another of the plurality of voltage waveforms is applied between the annulus electrode and the first iontophoresis electrode; and
a second iontophoresis electrode positioned between the second saline reservoir and the sweat sensing and collection unit and near an opening from the second microchannel to the skin, wherein the drug is iontophoretically delivered into the skin of the user when another of the plurality of voltage waveforms is applied from the first iontophoresis electrode to the second iontophoresis electrode, wherein the drug is configured to induce sweating.

14. The system of claim 13, wherein the controller is configured to:
determine whether the sufficient volume of sweat within the sweat chamber based on the electrical impedance of the gold electrode and the counter electrode;
if the volume of sweat within the sweat chamber is insufficient, then send a notification to a user, via a mobile device, to trigger the sweat stimulation unit; and
if the volume of sweat within the sweat chamber is sufficient, determine the chloride concentration of the sweat based on signals indicative of the electrochemical reaction of the gold molecules with the chloride ions when the other voltage waveform that causes chloride ions in the sweat to undergo an electrochemical reaction with gold molecules of the gold working electrode is applied.

15. A method comprising:
determining, by a system comprising a processor, whether an amount of sweat fills a chamber of a sweat collection unit, wherein a chloride sensing unit comprising a gold working electrode and a counter electrode is located within the chamber, and wherein the determining is based on an impedance between the gold working electrode and the counter electrode; and
when the impedance indicates that a sufficient amount of sweat fills the chamber,
delivering, by the system, a voltage waveform between the gold working electrode and the counter electrode within the chamber wherein molecules from the gold working electrode electrochemically react to the sweat in the chamber; and
detecting, by the system, a chloride concentration of the sweat in the chamber based on signals associated with the electrochemical reaction between the chloride ions and the molecules from the gold working electrode,
wherein information regarding the chloride concentration is used to determine one or more physiological conditions of the user based on the chloride concentration of the sweat in the chamber.

16. The method of claim 15, wherein the impedance is measured between the gold working electrode and the counter electrode.

17. The method of claim 16, wherein if the volume of sweat is not sufficient, then the method further comprises:
notifying, by the system, the user that sweat is insufficient and to trigger sweat stimulation unit of the system, wherein the user physically triggers the sweat stimulation unit.

18. The method of claim 17, further comprising:
releasing, by the system, a drug configured to stimulate sweat from a drug storage reservoir of the sweat stimulation unit via electroporation of an electrode covering an opening of the drug storage reservoir; and
wherein the drug is delivered through a saline pathway, released when the user physically triggers the sweat stimulation unit, into the skin of the user via iontophoresis between the electrode covering an opening of the drug storage reservoir, a first iontophoresis electrode, and a second iontophoresis electrode, wherein the drug induces the user to sweat.

19. The method of claim 17, further comprising:
selecting, by the system, one drug storage reservoir of a plurality of drug storage reservoirs to release a drug from to induce sweat.

20. The method of claim 15, wherein the chloride concentration is detected once, once a day, once an hour, or once a half hour.

21. The method of claim 15, wherein the system is a wearable device.
